(19) United States
(12) Patent Application Publication
(10) Pub. No.: US 2008/0167580 A1
(43) Pub. Date: Jul. 10, 2008

(54) Rehabilitation System

(75) Inventors: Arik Avni, Metar (IL); Lior Bar-Nes, Lehavim (IL); Ronit Friedman, Lehavim (IL); Dror Salah, Beer-Sheva (IL)

Correspondence Address: DARBY & DARBY P.C., P.O. BOX 770, Church Street Station, New York, NY, 10008-0770, US

(73) Assignee: ANDANTE MEDICAL DEVICES LTD., Omer (IL)

(21) Appl. No.: 11/910,699

(22) PCT Filed: Apr. 5, 2006
(86) PCT No.: PCT/IL06/00432

§ 371 Date: Dec. 28, 2007

Related U.S. Application Data
(60) Provisional application No. 60/668,738, filed on Apr. 5, 2005.

(51) Int. Cl. A61B 5/103; A43B 13/38

(52) U.S. Cl. 600/587; 36/43

(57) ABSTRACT

Apparatus (10) is provided for use with a shoe worn by a subject, the apparatus (10) including an insole system (20), adapted to be inserted into the shoe, the insole system (20) including a flexible insole (26) and at least one force sensor (28), adapted to generate a force measurement; and a control unit (24), adapted to receive the force measurement, and convert the force measurement to a weight measurement. Other embodiments are also described.

### CROSS-REFERENCES TO RELATED APPLICATIONS

The present application claims the benefit of U.S. Provisional Application 60/668,738 to Avni, filed Apr. 5, 2005, which is assigned to the assignee of the present application and is incorporated herein by reference, including the appendix thereof.

### FIELD OF THE INVENTION

The present invention relates generally to diagnostic and rehabilitation systems, and specifically to adaptive weight-bearing adaptive rehabilitation systems.

### BACKGROUND OF THE INVENTION

Physical therapy is necessary for orthopedic patients after fracture injury or joint replacement, for patients afflicted by neurological conditions such as cerebral vascular accident (CVA) and cerebral palsy (CP), for patients recovering from stroke, for patients subsequent to lower limb amputation, and for patients rehabilitating from sports injuries such as meniscal, ACL, or Achilles tendon tears.

During the rehabilitation of a simple fracture, the conventional medical approach has been a partial weight bearing (PWB) approach. The PWB approach is often recommended when a soft callus is evident on an x-ray typically taken 2 to 6 weeks after the injury. However, in the case of a complicated fracture, such as an intra-articular injury, weight bearing is typically completely prohibited until three months post-injury, and only then is the PWB approach prescribed. While weight bearing is essential for the bone building process, an overload can damage the bone fixation and the healing process. Hence, PWB as a prescription is problematic because it is subjective and the patient must decide by himself/herself how much to load the injured limb.

Several devices are known for assisting the therapist and patient in determining how much weight is being applied to a patient's lower extremity. Some of these devices warn the patient of an overload or an underload in the amount of pressure placed on the leg. For example, U.S. Pat. No. 5,619,186 to Schmidt et al., which is incorporated herein by reference, describes a foot weight alarm device including a foot-shaped insole device including resistive force sensors that fits inside the patient's shoe to warn the patient when the patient is putting too little or too much weight on a limited weight bearing foot. The foot weight alarm device also includes a shoe pouch which laces in the shoe, a foot weight alarm unit which fits in the shoe pouch and contains electronics that connects to the insole device, a data cable that is used by a health care professional to program the foot weight alarm unit, and a foot weight alarm calibration system used by the health care professional to program the foot weight alarm unit. The foot weight alarm unit measures the forces on each insole's sensors to compute the total force, and when the total force is below the target value, a low tone is produced by the foot weight alarm unit, while in the target zone a high tone is produced and above the target zone a two-tone warble is produced to inform the patient to take weight off the limb. Other features of the '186 patent's foot weight alarm device include an optimal data-logging feature that logs the time and maximum weight of each step for up to 16,000 steps. This feature provides the physician with the ability to review the patient's progress while at, and after leaving, the rehabilitation facility. In addition, a motion detector turns on the foot weight alarm device when the first step is taken by the patient, and power saving electronic circuitry turns the device off when there is no weight on the foot, thereby saving energy.

Hemiparesis due to stroke often results in spastic drop-foot (i.e., the loss of ability to dorsiflex the foot on the affected side). One approach to the management of spastic drop-foot is the prescription of an ankle foot orthosis (AFO), which holds the foot in a neutral position to prevent it from dragging during the swing phase of gait. An alternative approach is active stimulation of the dorsi and plantar flexors.

Electrical stimulation for correction of spastic drop foot in hemiplegia was first applied by Liberson and coworkers in 1961 (Liberson et. al., “Functional electrotherapy, stimulation of the peroneal nerve synchronized with the swing phase of gait of hemiplegic patients,” Arch Phys Med Rehab 42:101-105 (1961)). Surface electrodes were applied over the peroneal nerve at the head of the fibula. A stimulator worn around the waist was controlled by a footswitch in the heel of the shoe of the affected limb. When the patient lifted the heel to take a step, the stimulator was activated. Stimulation was stopped when the heel contacted the ground. This peroneal stimulator system produces dorsiflexion and eversion of the foot during the swing phase of gait (Granat et al., “Peroneal stimulator: evaluation for the correction of spastic drop foot in hemiplegia,” Arch Phys Med Rehab 77:19-24 (1996)). This system and other electrical stimulation systems are dependent on a sensor system to accurately sense when and to measure how much force is being applied to a region or regions of the foot. Both of these articles are incorporated herein by reference.

There are a number of insole foot force sensing devices currently used for measuring force on the foot. For example, U.S. Pat. No. 4,745,930 to Confer, which is incorporated herein by reference, describes a flexible force sensing insole which incorporates multiple electrical switches which close after a certain threshold level of force is imposed on the insole. U.S. Pat. No. 5,033,291 to Podoloff et al., which is incorporated herein by reference, describes a force sensing device which uses a plurality of intersecting electrodes. The electrodes act as open circuit switches at each intersection which close when force is applied to the insole at that intersection location. The resistance between the two electrodes varies with the amount of force applied. U.S. Pat. No. 4,426,884 to Polchaninoff, which is incorporated herein by reference, describes a flexible force sensor which acts as an open circuit, closing with the application of force on the sensor and having resistance that varies with the amount of force.

Foot force measurement devices typically convert mechanical force into a suitable signal medium, usually electrical signals. The devices thus can be conveniently categorized according to the type of sensor used to convert changes in mechanical force to changes in electrical signals. These types of sensors include switches, strain gauge sensors that respond to mechanical deformation, single direct electronic force sensors, multiple direct electronic force sensors with random spacing, and multiple direct electronic force sensors with regular spacing.

U.S. Pat. No. 4,813,436 to Au, which is incorporated herein by reference, describes a motion analysis system that incorporates markers which are secured at various joints of a subject's body, and pressure-sensitive shoes or insoles which are worn by the subject. The subject is caused to perform motion such as walking or running. While performing this motion, the subject is televised by means of two video cameras. A display is provided which indicates the pressure applied to the subject's foot while performing the motion, as measured by the pressure-sensitive insoles. The remaining data supplied by the video cameras is processed to present various displays showing the gait, the angular position of the various joints of the subject, and various other information indicative of the particular walking characteristics of the subject. The data produced and processed by the system enables a practitioner to compare the subject's walling gait to that of a normal user.

U.S. Pat. Nos. 4,734,034 and 4,856,993 to Maness et al., which are incorporated herein by reference, describe a contact sensor for detecting points on a grid where the sensor is being contacted on opposing sides by teeth surfaces or other contacting points. The contact sensor includes two sets of parallel electrodes which are each formed of a thin, flexible supporting sheet. The electrodes are coated with a thin, resistive coating. Two such electrode structures are oriented at approximately right angles to create a grid where the intersecting electrodes cross separated by the resistive coatings. The resistive coatings may be made from conventional resistive inks and are optionally separated by a separation material, such as talcum or mesh. In the absence of an external force, the material between the electrodes sets provides a high resistance between intersecting electrodes. The composition of the intermediate layer results in a structure which provides a “switching” effect such that the resistance between electrodes is very high where there is no external pressure and changes to a comparatively low value at locations where external pressure is applied by two contacting points or surfaces.

U.S. Pat. No. 3,881,496 to Vredenbregt et al., which is incorporated herein by reference, describes techniques for electrically stimulating leg muscles using an air-filled chamber located in the sole of the shoe beneath the ball of the foot. The chamber is coupled through an air channel or a thin hose and a diaphragm to a microswitch located in the heel. The switch activates an electric pulse generator in synchronism with the normal walking pattern.

U.S. Pat. No. 3,974,491 to Sipe, which is incorporated herein by reference, describes a sensor having a fluid-filled chamber that is a continuous, resilient tube having a circular cross section. The tube is coiled under the heel and the sole of a patient's foot inside a sponge rubber footpad. The footpad is placed between adhesive sheets of flexible, dimensionally stable material such as rubber-coated fabric.

U.S. Pat. No. 3,791,375 to Pfeiffer, which is incorporated herein by reference, describes a remote displacement measuring device that is connected to two units, a heel unit and a toe unit, located in the insole. The units deflect and change their volume in accordance with the amount of load placed thereon. The displacement measuring device is signaled with an electrical alarm to indicate when a predetermined load on the units is reached. The displacement measuring device consists of a single sensor such as a bellows that measures the combined total displacement from both the heel and the toe unit.

U.S. Pat. No. 6,273,863 to Avni et al., which is assigned to the assignee of the present application and is incorporated herein by reference, describes a portable, self-learning adaptive weight bearing monitoring system for personal use during rehabilitation of orthopedic patients with fractures of the lower extremities. The system includes a flexible insole which is worn inside the shoe. The insole includes pressure and/or force sensor that measure the Ground Reaction Force (GRF) applied at key bearing points under the foot or other portions of the patient's lower extremity. The sensors are, in turn, connected through an A/D converter to a CPU that is connected so as to drive a stimulator that delivers closed-loop sensory stimulation (electrical, mechanical, and/or audio) as feedback to encourage the patient to load the optimal target weight for the limb for which the weight bearing force is being measured. Accurate real-time monitoring of the weight bearing during physical rehabilitation is also provided, and, through the use of closed-loop sensory stimulation, the patient is given continuous feedback for improving rehabilitation.

PCT Publication WO 04/008095 to Avni et al., which is assigned to the assignee of the present application and is incorporated herein by reference, describes a force sensor system for use in monitoring weight bearing on a location. The force sensor system comprises at least one a foot force sensor, a palm force sensor, and a knee force sensor. The foot force sensor comprises a flexible insole containing a plurality of inflatable pockets that are inflated with air or liquid. The palm force sensor and knee force sensor each comprise a wrap to be worn around the palm and knee, respectively. Each wrap comprises a pocket. Each pocket is connected to a tube that, in turn, connects with a pressure sensor and a connector coupling that is remote from the pocket. Each coupling contains a valve. The valve opens to allow inflation and deflation of each inflatable pocket. The pressure sensors measure the air or liquid pressure within each of the inflatable pockets, and convert the corresponding pressure signal into a suitable output signal medium, usually electrical signals. The output signal from the sensors provides accurate real time input data to a weight bearing biofeedback system or to control a stimulator for activation of an electronic orthosis to normalize dynamic gait patterns.

PCT Publication WO 01/36051 to Avni, which is assigned to the assignee of the present application and is incorporated herein by reference, describes a portable, self-learning adaptive weight bearing monitoring system for personal use during rehabilitation of neurological disorders and orthopedic lower limb injuries. The system includes a flexible insole or pad which includes at least one pressure and/or force sensor that measures the weight force applied to at least two monitored locations of at least one of the patient's limbs. The sensors are, in turn, connected through an A/D converter to a CPU that compares the distribution of weight on each monitored location of at least one limb to a target weight distribution. The target weight distribution is preferably based on subjective and objective parameters unique to the patient and the injury of the patient. The CPU is connected so as to drive a stimulator that delivers closed-loop sensory stimulation (visual, mechanical vibration, and/or audio) as feedback to encourage the patient to distribute weight more evenly on all monitored locations of at least one limb. Accurate real-time monitoring of the weight bearing during physical rehabilitation is also provided, and, through the use of closed-loop sensory stimulation, the patient is given continuous feedback for improving rehabilitation.

U.S. Pat. No. 6,360,597 to Hubbard, Jr., which is incorporated herein by reference, describes a gait analysis system that includes a shoe insert for use in a shoe worn by a subject while walking as part of a process of collecting gait data. The insert has force-sensing sensors distributed to define a sensing aperture, and each sensor provides an electrical output signal. Processing apparatus is communicatively coupled with the sensors. The processing apparatus calculates a gait line represented by a series of points, wherein each point is calculated as a spatially-weighted average of samples of the sensor output signals over the sensing aperture. The processing apparatus includes a portable telemetry transmitter worn by the subject. The transmitter is connected to the sensors to receive the sensor output signals, and transmits a radio signal carrying the sensor information. A stationary receiver receives the sensor information in a transmission from the transmitter, and provides the sensor information to a personal computer or similar workstation.

U.S. Pat. No. 6,611,789 to Darley, which is incorporated herein by reference, describes a method including determining, with at least one device supported by a user while the user is in locomotion on foot on a surface, an amount of force exerted by at least one foot of the user on the surface during at least one footstep taken by the user. In another embodiment, a method includes: (a) with at least one sensor supported by a user, monitoring movement of the user while the user is in locomotion on foot; and (b) determining a cadence of the user based upon an output of the at least one sensor. In another embodiment, a method includes: (a) with at least one sensor supported by a user while the user is in locomotion on foot, monitoring movement of the user while the user is in locomotion on foot; and (b) determining a stride length of the user during at least one footstep taken by the user based upon an output of the at least one sensor. In one embodiment, a display has simultaneously displayed thereon at least one determined performance parameter of the user (e.g., pace) and at least one determined variable physiological parameter of the user (e.g., heart rate).

U.S. Pat. No. 6,493,652 to Ohlenbusch et al., which is incorporated herein by reference, describes a method including, in response to movement of a user during at least one footstep taken by the user, generating a signal that experiences changes during a time period that the foot is airborne during the at least one footstep. At least one change in the signal generated after the foot has become airborne and before the foot contacts a surface is identified that is indicative of the foot being airborne during the at least one footstep. In another embodiment, a method includes generating a signal in response to movement of a user during at least one footstep taken by the user. The signal is monitored to determine when the signal has experienced a minimum degree of smoothness for at least a given period of time. In response to determining that the signal has experienced the minimum degree of smoothness for at least the given period of time, the foot of the user is identified as being airborne.

U.S. Pat. No. 5,406,719 to Potter, which is incorporated herein by reference, describes a cushioning element for use in a shoe. The cushioning element includes four fluid-filled support chambers which are compressible but not collapsible, and which are disposed at different locations throughout the midsole of the shoe. The element also includes four variable volume fluid reservoir chambers which are collapsible to reduce the volume thereof. The reservoir chambers are controllably linked in fluid communication with the support chambers so as to be selectively in fill communication with or isolated from the support chambers. By selectively isolating one or more of the reservoir chambers from one or more of the support chambers, and collapsing the isolated chamber, fluid may be moved from one support chamber to another at a different location, thereby increasing the stiffness of the midsole at a selected location.

U.S. Pat. No. 6,430,843 to Potter et al., which is incorporated herein by reference, describes an article of footwear with a dynamically-controlled cushioning system. The cushioning system includes a sealed, fluid-filled bladder formed with a plurality of separate cushioning chambers, and a control system. The control system, which includes pressure sensors and valves, controls fluid communication between the chambers to dynamically adjust the pressure in the cushioning chambers for various conditions such as the activity that the footwear is used in, the weight of the individual and the individual's running style. Certain adjustments can be made while the footwear is in use.

US Patent Application Publication 2003/0009913 to Potter et al., which is incorporated herein by reference, describes an article of footwear with a dynamically-controlled cushioning system. The cushioning system includes a sealed, fluid-filled bladder formed with a plurality of separate cushioning chambers, and a control system. The control system, which includes pressure sensors and valves, controls fluid communication between the chambers to dynamically adjust the pressure in the cushioning chambers for various conditions such as the activity that the footwear is used in, the weight of the individual, and the individual's running style. Certain adjustments can be made while the footwear is in use.

U.S. Pat. No. 6,298,314 to Blackadar et al., which is incorporated herein by reference, describes methods for monitoring movement of a person, including using a sensor to generate a signal in response to movement of the person. In one embodiment, a characteristic in the signal is identified that indicates the person is walking or running and, in response to identifying the characteristic, a timer is started. In another embodiment, after the person has begun walking or running, a characteristic in the signal is identified that indicates the person has ceased walling or running and, in response to identifying the characteristic, an action is taken. In another embodiment, a characteristic in the signal is identified that is indicative of a foot of the person being in motion and, in response to identifying the characteristic, a timer is started. In another embodiment, after a foot of the person has been in motion, a characteristic in the signal is identified that is indicative of the foot ceasing to be in motion and, in response to identifying the characteristic, an action is taken. In another embodiment, in response to identifying that the person is not walling or running, a characteristic in the signal is identified that indicates the person has begun walking or running and, in response to identifying the characteristic, an action is taken. In another embodiment, in response to identifying that a foot of the person is stationary, a characteristic in the signal is identified that indicates the foot is in motion and, in response to identifying the characteristic, an action is taken.

U.S. Pat. No. 5,253,435 to Auger et al., which is incorporated herein by reference, describes a bladder assembly for an athletic shoe having at least first and second chambers. The chambers are independently and separately pressure adjustable by the user to conform to different concavity areas of his foot, such as the arch, ankle and metatarsal areas, to thereby enhance fit, comfort and athletic performance. Both chambers are inflatable by the same articulated on-board pump and deflatable by the same on-board depressible plunger. A dial on the lateral side of the upper allows the user to select which of the chambers is to be pressure adjusted, that is, which of the chambers is in pressure communication with the pump and the plunger. When the dial is in a neutral position, accidental inflation or deflation of either chamber is prevented.

U.S. Pat. No. 5,107,854 to Knotts et al., which is incorporated herein by reference, describes an orthopedic exercise chamber such as a slipper including a light-weight, self-contained limb load monitor is disclosed. The limb load sensor circuit provides extended service life for the miniature power supply that is included in the slipper, thereby making the slipper suitable for outpatient use. A fluid-filled plantar chamber that supports the entire load borne by the patient's foot is connected to the sensor circuit, thereby providing improved monitoring of the load being carried by the leg or foot that must be protected from excessive loading.

U.S. Pat. No. 6,646,643 to Templeman, which is incorporated herein by reference, describes techniques for interfacing locomotive 3D movements of a user to a reference in a virtual or remote environment are provided. Initially, a 3D motion of a body portion of a user is sensed as the user takes a gestural pace. This sensing includes the determining of a beginning and an end of the gestural pace taken by the user, the determining of a 3D direction characteristic of the body portion motion during the gestural pace, and the determining of a 3D extent characteristic of the body portion motion during the gestural pace. Next, a 3D direction and extent of motion in the environment corresponding to the determined direction and extent characteristics of the gestural pace is computed. Finally, the computed 3D motion is used to move the reference in the environment.

U.S. Pat. No. 6,539,336 to Vock et al., which is incorporated herein by reference, describes techniques for detecting the loft time, speed, power and/or drop distance of a vehicle, such as a sporting vehicle, during activities of moving and jumping. A loft sensor detects when the vehicle leaves the ground and when the vehicle returns to the ground. A controller subsystem converts the sensed information to determine a loft time. A display shows the recorded loft time to a user of the system. In addition, a speed sensor can detect the vehicle's speed for selective display to the user. A power sensing section informs the user of expended energy, which can be compared to other users. A drop distance sensing unit informs the user of the peak height of a jump, during an airtime. Gaming on the internet is facilitated to connect worldwide sport enthusiasts. The system can be integrated within a shoe and may thus be used by a jogger to evaluate different running shoes. Alternatively, when calibrated, the system is useful to joggers who can gate it to serve as a pedometer. The addition of a capacitor sensor in the heel helps determine average weight. A sensor for skin resistivity may additionally be used to record pulse. The shoe can also record the state of aerobic health for the jogger.

U.S. Pat. No. 6,398,740 to Lavery et al., which is incorporated herein by reference, describes techniques for monitoring items of vital health information including temperature of the plantar aspects of the foot of the human, body weight, blood pressure, pulse rate, blood glucose level and blood oxygen level. The apparatus includes a platform on which the user stands. Included on the platform are a set of heat sensitive signal generating devices. The temperature at predetermined locations on the plantar aspects of the human foot are determined by the signals obtained from the individual heat sensitive, signal generating probes. Other items of vital health information may be obtained by other sensors on the apparatus.

U.S. Pat. No. 5,642,096 to Leyerer et al., which is incorporated herein by reference, describes a shoe for prevention of ulcers in the feet of diabetes patients. The shoe includes a sensor disposed in a contained liquid mass of a hydrocell carried in the shoe inner sole, the sensor being one that detects both pressure and temperature values to which the patient's feet are exposed. The sensor includes a bridge circuit comprised of four piezoresistors arranged in two diagonally arrayed pairs, the resistance of one pair of resistors increasing and the resistance of the second pair decreasing in the presence of an increase in the pressure condition in the hydrocell, the resistance of all the resistors increasing or decreasing responsive to respective increases and decreases of temperature in the hydrocell. Outputs from the bridge circuit indicative of respective pressure and temperature values are acquired by a warning signal generator to operate same to generate a patient discernible warning signal that indicates to the patient a need to take action to avoid continuance of exposure to the condition. A grid array sensor detects localized pressure changes on the bottom of the foot by reducing the resistance between conductors present at the location of the increases pressure. The decreased resistance causes an increase in current flow between the conductors which is detected by a processor which in turn provides an indication of the increased pressure condition.

U.S. Pat. No. 5,736,656 to Fullen et al., which is incorporated herein by reference, describes techniques for continuously measuring the timing and distribution of forces exerted against the entire surface of a quadruped's foot, thereby providing a diagnostic tool to assist in measuring the ambulatory functions of an animal. The animal wears a force sensing device, either without a shoe within an attached boot, or incorporated in a modified conventional horseshoe which fits over the hoof of the animal. The apparatus can further include a remote sensing and feedback function which allows the processing, storing, display and feedback of data indicative of the ambulatory functions of the animal at one or more remote locations.

US Patent Application 2004/0122483 to Nathan et al., which is incorporated herein by reference, describes a gait modulation system for improving lower-limb function of a patient having neuromuscular impairment of the lower limbs. The system includes: (a) a sensor associated with a lower limb, for transducing a parameter related to the lower limb, so as to obtain gait information related to at least one gait event within a gait cycle; (b) a neuroprosthesis device including: (i) an electrode array operatively connected to an impaired lower limb, for performing functional electrical stimulation (FES) of at least one muscle of the impaired lower limb; (c) a muscle stimulator operatively connected to the electrode array, for supplying a muscle stimulation output to the array, and (d) a microprocessor operatively connected to the sensor, the microprocessor for processing the gait information and for controlling the muscle stimulation output via the muscle stimulator. The microprocessor is configured to control the autonomous reduction based on the gait information.

US Patent Application 2003/0009308 to Kirtley, which is incorporated herein by reference, describes a combination of sensors, including solid-state gyros and force-sensitive resistors, mounted in an insole suitable for insertion into a shoe. Data from the sensors is recorded by an in situ Programmable Interface Controller, logged into on-board EEPROM/Flash memory, and relayed to a base station computer via a miniature telemetry transmitter triggered by RFID tagging. Software then uses this data to compute the cadence and ankle power of the subject, as well as other parameters, in order to analyze and assess the gait and activity of the subject. The device is described as being useful for treatment of various disorders by biofeedback, sounding of alarms, control of movement of air/fluid between sacs by valves.

A brochure by MarketReach America describes the SmartStep® monitoring and biofeedback system (Andante Medical Devices Ltd., Omer, Israel) as a portable, miniature monitoring and biofeedback system for patients undergoing rehabilitation treatment.

The ForceGuard® Weight-Bearing System (Smith & Nephew, Germantown, Wis.) is an electronic foot pad pressure sensor that emits an audio signal when applied pressure exceeds a set limit.

The PedAlert® weight-bearing monitor is marketed for patients with hip fractures, hip replacements, foot ulcers, and other conditions that require weight-bearing limitation or weight transfer training. The monitor includes a cast shoe including a membrane sensor which monitors the weight placed on the lower limb. A warning tone signals when the threshold is met or exceeded. Some models monitor the entire foot, while other models additionally independently monitor the forefoot and hindfoot.

The F-Scan® system (Tekscan, Inc., South Boston, Mass.) provides bipedal plantar pressure and force measurements on the feet, using paper-thin reusable sensors placed in the shoes. The system detects, displays, and records plantar pressures and forces.

The Pedar system (Novel GmbH, Munich, Germany) is a pressure distribution measuring system for monitoring local loads between the shoe and the foot.

### SUMMARY OF THE INVENTION

In embodiments of the present invention, a portable rehabilitation system comprises at least one pressure and/or force sensor that measures the force applied to a limb of a subject, a stimulator that provides feedback to encourage the subject to load an optimal target weight for the limb, and a control unit that receives the force measurements and drives the stimulator responsive thereto. For some applications, the stimulator comprises an audio stimulator (e.g., a tone generator), a tactile stimulator (e.g., a vibrating unit), and/or a visual stimulator (e.g., a series of LED's, and/or a computer monitor). Typically, the limb includes a foot of the subject, and the system comprises a flexible insole which comprises the sensor or a portion thereof, and is adapted to be worn inside a shoe of the subject. The system thus provides real-time monitoring of weight bearing during physical rehabilitation, and, through the use of closed-loop sensory stimulation, provides the subject with continuous feedback and/or feedforward data for improving rehabilitation.

In some embodiments of the present invention, the control unit comprises a user interface, which comprises a display. The control unit is configured to display parameters in a limited number of parameter categories, typically no more than four categories, which are typically selected because they are of particular practical usefulness to a physiotherapist using the system. The control unit typically does not display parameters in additional parameter categories, such as those generally displayed in research-oriented rehabilitation systems, which are not of particular usefulness to the physiotherapist.

There is therefore provided, in accordance with an embodiment of the present invention, apparatus for use with a shoe worn by a subject, the apparatus including:
an insole system, adapted to be inserted into the shoe, the insole system including a flexible insole and at least one force sensor, adapted to generate a force measurement; and
a control unit, adapted to receive the force measurement, and convert the force measurement to a weight measurement.

For some applications, the force measurement is expressed as a digital value, and the force sensor is adapted to convert a measured analog signal into the digital force measurement.

For some applications, the control unit includes a memory, in which a look-up table is stored, the look-up table containing force-to-weight conversion values for a plurality of force measurement values, and the control unit is adapted to use the look-up table to convert the force measurement to the weight measurement.

For some applications, the force sensor is adapted to generate separate force measurements for hindfoot and forefoot regions of the insole, and the control unit is adapted to convert the hindfoot force measurement to a hindfoot weight measurement, and the forefoot force measurement to a forefoot weight measurement.

In an embodiment, the control unit is adapted to convert the force measurement to the weight measurement at least in part responsively to a shoe/foot characteristic. For some applications, the control unit is adapted to convert the force measurement to the weight measurement at least in part responsively to a characteristic of the shoe selected from the group consisting of: a type of the shoe, and a size of the shoe. Alternatively or additionally, the control unit is adapted to convert the force measurement to the weight measurement at least in part responsively to an indication of whether the subject is an amputee who is fitted with a prosthesis that is inserted in the shoe. Further alternatively or additionally, the control unit is adapted to convert the force measurement to the weight measurement at least in part responsively to an indication of whether an orthotic insert is inserted in the shoe.

In an embodiment, the force sensor includes a pressure sensor, adapted to measure a pressure in the insole and generate the force measurement responsively to the measured pressure. For some applications, the pressure sensor includes at least two pressure sensors, positioned remotely from the insole; the insole is shaped so as to define at least two independent chambers inflatable with a substance selected from the group consisting of: a gas and a liquid; and the insole system includes at least two tubes, each of which is in fluid communication with a respective one of the chambers and with a respective one of the pressure sensors. For some applications, the insole is shaped so as to define exactly two independent chambers that are operatively coupled to respective ones of the pressure sensors. For some applications, the insole is shaped so as to define a first one of the chambers in a hindfoot region of the insole, and a second one of the chambers in a forefoot region of the insole.

For some applications, the control unit is adapted to convert the force measurement to the weight measurement at least in part responsively to an indication of whether the user is stepping or standing still. For some applications, the control unit includes a memory, in which a look-up table is stored, the look-up table containing, for a plurality of force measurement values, stepping force-to-weight conversion values and standing-still force-to-weight conversion values, and the control unit is adapted to use the look-up table to convert the force measurement to the weight measurement.

There is further provided, in accordance with an embodiment of the present invention, apparatus for use with a shoe worn by a subject, the apparatus including:
an insole system, adapted to be inserted into the shoe, the insole system including a flexible insole and at least one force sensor, adapted to generate a force measurement; and
a control unit, adapted to receive the force measurement, and utilize the force measurement in a calculation that is performed at least in part responsively to a shoe/foot characteristic.

For some applications, the force measurement is expressed as a digital value, and the force sensor is adapted to convert a measured analog signal into the digital force measurement.

For some applications, the shoe/foot characteristic includes an indication of whether the subject is an amputee fitted with a prosthesis that is inserted in the shoe, and the control unit is adapted to utilize the force measurement in the calculation that is performed at least in part responsively to the indication. Alternatively or additionally, the shoe/foot characteristic includes an indication of whether an orthotic insert is inserted in the shoe, and the control unit is adapted to utilize the force measurement in the calculation that is performed at least in part responsively to the indication.

In an embodiment, the shoe/foot characteristic includes a characteristic of the shoe, and the control unit is adapted to utilize the force measurement in the calculation that is performed at least in part responsively to the characteristic of the shoe.

For some applications, the characteristic of the shoe includes a size of the shoe, and the control unit is adapted to perform the calculation at least in part responsively to the shoe size. Alternatively or additionally, the characteristic of the shoe includes a type of the shoe, and the control unit is adapted to perform the calculation at least in part responsively to the shoe type. Further alternatively or additionally, the characteristic of the shoe includes a size of the shoe and a type of the shoe, and the control unit is adapted to perform the calculation at least in part responsively to the shoe size and the shoe type.

For some applications, the control unit is adapted to convert the force measurement to a weight measurement at least in part responsively to the shoe/foot characteristic. For some applications, the shoe/foot characteristic includes a size of the shoe, and the control unit is adapted to convert the force measurement to the weight measurement at least in part responsively to the shoe size. Alternatively or additionally, the shoe/foot characteristic includes a type of the shoe, and the control unit is adapted to convert the force measurement to the weight measurement at least in part responsively to the shoe type. Further alternatively or additionally, the shoe/foot characteristic includes a size of the shoe and a type of the shoe, and the control unit is adapted to convert the force measurement to the weight measurement at least in part responsively to the shoe size and the shoe type.

For some applications, the control unit includes a memory, in which a look-up table is stored, the look-up table containing force-to-weight conversion values for a plurality of force measurement values and a plurality of values of the shoe/foot characteristic, and the control unit is adapted to use the look-up table to convert the force measurement to the weight measurement.

For some applications, the force sensor is adapted to generate separate force measurements for hindfoot and forefoot regions of the insole, and the control unit is adapted to utilize the hindfoot force measurement and the forefoot force measurement in the calculation that is performed at least in part responsively to the shoe/foot characteristic. For some applications, the control unit is adapted to convert, at least in part responsively to the shoe/foot characteristic, the hindfoot force measurement to a hindfoot weight measurement, and the forefoot force measurement to forefoot weight measurement. For some applications, the control unit includes a memory, in which a look-up table is stored, the look-up table containing, for a plurality of force measurement values and a plurality of values of the shoe/foot characteristic, hindfoot force-to-weight conversion values and forefoot force-to-weight conversion values, and the control unit is adapted to use the look-up table to convert the hindfoot and forefoot force measurements to the hindfoot and forefoot weight measurements, respectively.

For some applications, the control unit is adapted to perform the calculation at least in part responsively to an indication of whether the user is stepping or standing still. For some applications, the control unit is adapted to convert the force measurement to a weight measurement, at least in part responsively to the indication of whether the user is stepping or standing still. For some applications, the control unit includes a memory, in which a look-up table is stored, the look-up table containing, for a plurality of force measurement values and a plurality of values of the shoe/foot characteristic, stepping force-to-weight conversion values and standing-still force-to-weight conversion values, and the control unit is adapted to use the look-up table to convert the force measurement to the weight measurement.

In an embodiment, the force sensor includes a pressure sensor, adapted to measure a pressure in the insole and generate the force measurement responsively to the measured pressure. For some applications, the pressure sensor includes at least two pressure sensors, positioned remotely from the insole; the insole is shaped so as to define at least two independent chambers inflatable with a substance selected from the group consisting of: a gas and a liquid; and the insole system includes at least two tubes, each of which is in fluid communication with a respective one of the chambers and with a respective one of the pressure sensors. For some applications, the insole is shaped so as to define exactly two independent chambers that are operatively coupled to respective ones of the pressure sensors. For some applications, the insole is shaped so as to define a first one of the chambers in a hindfoot region of the insole, and a second one of the chambers in a forefoot region of the insole.

There is yet further provided, in accordance with an embodiment of the present invention, apparatus for use with a shoe worn by a subject, the apparatus including:
an insole system, adapted to be inserted into the shoe, the insole system including a flexible insole, and at least two force sensors adapted to sense force at respective locations of the insole, and, responsive thereto, to generate respective force measurements;
a stimulator, adapted to provide feedback to the subject; and
a control unit, adapted to:
   receive the force measurements,
   perform an analysis of a gait of the subject at least in part responsively to the force measurements, and
   responsively to the gait analysis, drive the stimulator to configure the feedback to guide the subject to modify one or more respective forces applied by the subject to a subset of the respective locations of the insole, the subset containing fewer than all of the locations of the insole.

There is still further provided, in accordance with an embodiment of the present invention, apparatus for use with a shoe worn by a subject, the apparatus including:
an insole system, adapted to be inserted into the shoe, the insole system including a flexible insole and at least one force sensor, adapted to generate a force measurement; and
a portable control unit, adapted to be physically coupled to and carried on a limb of the subject, the control unit including a memory, in which a look-up table is stored, the look-up table containing force-to-weight conversion values for a plurality of values of at least one shoe/foot characteristic, and the control unit adapted to:
receive an indication of a value of the shoe/foot characteristic,
receive the force measurement, and
use the look-up table to convert the force measurement to a weight measurement, at least in part responsively to the value of the shoe/foot characteristic.

For some applications, the force measurement is expressed as a digital value, and the force sensor is adapted to convert a measured analog signal into the digital force measurement.

For some applications, the shoe/foot characteristic includes a type of the shoe, and the look-up table contains the force-to-weight conversion values for a plurality of shoe types. Alternatively or additionally, the shoe/foot characteristic includes a size of the shoe, and the look-up table contains the force-to-weight conversion values for a plurality of shoe sizes. Further alternatively or additionally, the shoe/foot characteristic includes a type of the shoe and a size of the shoe, and the look-up table contains the force-to-weight conversion values for a plurality of shoe types and shoe sizes.

For some applications, the shoe/foot characteristic includes an indication of whether the subject is an amputee fitted with a prosthesis that is inserted in the shoe, and the look-up table contains force-to-weight conversion values for an individual indicated to be an amputee fitted with a prosthesis that is inserted in the shoe and for an individual not indicated to be an amputee fitted with a prosthesis that is inserted in the shoe. Alternatively or additionally, the shoe/foot characteristic includes an indication of whether an orthotic insert is inserted in the shoe, and the look-up table contains force-to-weight conversion values for an orthotic insert being inserted in the shoe and for an orthotic insert not being inserted in the shoe.

There is also provided, in accordance with an embodiment of the present invention, a method for calibrating a flexible insole, the method including:
inserting the insole into a shoe;
inserting a foot of a subject into the shoe after the insole has been inserted;
weighing, one or more times, the shoe while the subject is applying weight to the shoe, so as to generate a set of one or more measured weight values;
while weighing the shoe, measuring a force applied by the foot on the insole, so as to generate a set of one or more measured force values; and
responsively to the measured weight values and the measured force values, generating a look-up table containing force-to-weight conversion values applicable to the shoe.

For some applications, weighing the shoe and measuring the force applied by the foot include weighing the shoe and measuring the force applied by the foot a first number of one or more times, and generating the look-up table includes generating the look-up table containing a second number of the force-to-weight conversion values, the second number greater than the first number. For some applications, the first number is exactly one time.

For some applications, generating the look-up table includes fitting the measured weight and force values to a predetermined curve, in order to generate the second number of the force-to-weight conversion values.

For some applications, the method includes:
generating for a calibration shoe a calibration-shoe table indicating force-to-weight conversion values, the calibration-shoe table including at least two portions, the at least two portions including a calibration-shoe high-force portion and a calibration-shoe low-force portion, the calibration-shoe high-force portion including higher force values than the calibration-shoe low-force portion; and
identifying a numerical relationship relating values in the calibration-shoe high-force portion to values in the calibration-shoe low-force portion, and
the look-up table includes at least two portions, the at least two portions including a look-up table high-force portion and a look-up table low-force portion, the look-up table high-force portion including higher force values than the look-up table low-force portion, and
generating the look-up table includes setting values in the look-up table responsively to the identified relationship between the values in the calibration-shoe high-force portion and the values in the calibration-shoe low-force portion.

For some applications, the numerical relationship includes a relationship between (a) a characteristic slope of the values in the calibration-shoe high-force portion and (b) a characteristic slope of the values in the calibration-shoe low-force portion, and generating the look-up table includes generating the look-up table responsively to the relationship between the characteristic slopes.

In an embodiment, generating the look-up table includes: providing a look-up table pre-populated with a plurality of force-to-weight conversion values applicable to a typical foot and a typical shoe; and correcting the conversion values of the pre-populated look-up table responsively to the measured weight values and the measured force values. For some applications, weighing includes instructing the subject, during each of the one or more times, to apply a respective weight to the shoe equal to a respective weight value of one of the force-to-weight conversion values in the pre-populated look-up table. For some applications, weighing includes weighing between one and three times.

There is additionally provided, in accordance with an embodiment of the present invention, a method for calibrating a flexible insole shaped so as to define at least two independent chambers inflatable with a gas, including:
inserting the insole into a shoe;
inserting a foot of a subject into the shoe after the insole has been inserted;
measuring respective pressures in each of the chambers after the foot has been inserted, while weight of the subject is not applied to the shoe; and
inflating each of the chambers until respective desired pressures are obtained in the chambers.

There is still additionally provided, in accordance with an embodiment of the present invention, apparatus for use with a shoe worn by a subject, the apparatus including:
an insole system, adapted to be inserted into the shoe, the insole system including a flexible insole and at least one force sensor, adapted to generate first and second force measurements for first and second regions of the insole, respectively, the first and second regions corresponding to first and second regions of a sole of the shoe, respectively; and
a control unit, adapted to:
receive the first and second force measurements,
generate a first output responsively to the first force measurement surpassing a first minimum threshold value indicative of ground contact of the first region of the sole, and
generate a second output, different from the first output, responsively to the second force measurement surpassing a second minimum threshold value indicative of ground contact of the second region of the sole.

For some applications, the first and second minimum threshold values are each indicative of a threshold weight equal to between 5% and 10% of a weight of the subject.

For some applications, the first and second regions of the insole include hindfoot and forefoot regions of the insole, respectively, and the at least one force sensor is adapted to generate the first and second force measurements for the hindfoot and forefoot regions of the insole, respectively.

For some applications, the first and second outputs include first and second audible outputs, respectively, and the control unit is adapted to generate the first and second audible outputs.

There is further provided, in accordance with an embodiment of the present invention, apparatus for use with a shoe worn by a subject, the apparatus including:
an insole system, adapted to be inserted into the shoe, the insole system including a flexible insole and at least one force sensor, adapted to generate first and second force measurements for first and second regions of the insole, respectively; and
a control unit, adapted to:
receive a measured sequence of the first and second force measurements,
compare the measured sequence with a predetermined sequence, and
generate a correspondence output responsively to a determination that the measured sequence corresponds to the predetermined sequence.

For some applications, the control unit is adapted to generate a non-correspondence output responsively to a determination that the measured sequence does not correspond to the predetermined sequence.

For some applications, the correspondence output includes an audible correspondence output, and the control unit is adapted to generate the audible correspondence output.

For some applications, first and second regions of the insole include hindfoot and forefoot regions of the insole, respectively, and the at least one force sensor is adapted to generate the first and second force measurements for the hindfoot and forefoot regions of the insole, respectively. For some applications, the predetermined sequence is indicative of heel-toe walking, and the control unit is adapted to generate the correspondence output responsively to the determination that the measured sequence is indicative of the heel-toe walking.

There is also provided, in accordance with an embodiment of the present invention, a method for use with a shoe worn by a subject, the method including:
inserting a flexible insole into the shoe;
generating a force measurement of a force applied to the insole; and
converting the force measurement to a weight measurement.

There is additionally provided, in accordance with an embodiment of the present invention, a method for use with a shoe worn by a subject, the method including:
inserting a flexible insole into the shoe;
generating a force measurement of a force applied to the insole; and
utilizing the force measurement, performing a calculation at least in part responsively to a shoe/foot characteristic.

There is still additionally provided, in accordance with an embodiment of the present invention, a method for use with a shoe worn by a subject, the method including:
inserting a flexible insole into the shoe;
sensing force at respective locations of the insole, and, responsive thereto, to generating respective force measurements;
providing feedback to the subject;
performing an analysis of a gait of the subject at least in part responsively to the force measurements; and
responsively to the gait analysis, configuring the feedback to guide the subject to modify one or more respective forces applied by the subject to a subset of the respective locations of the insole, the subset containing fewer than all of the locations of the insole.

There is further provided, in accordance with an embodiment of the present invention, a method for use with a shoe worn by a subject, the method including:
inserting a flexible insole into the shoe;
generating a force measurement of a force applied to the insole;
physically coupling a portable control unit to a limb of the subject, such that the control unit is carried on the limb;
receiving, by the control unit, an indication of a value of at least one shoe/foot characteristic; and
converting, by the control unit, the force measurement to a weight measurement, at least in part responsively to the value of the shoe/foot characteristic, using a look-up table containing force-to-weight conversion values for a plurality of values of the at least one shoe characteristic.

There is still further provided, in accordance with an embodiment of the present invention, a method for use with a shoe worn by a subject, the method including:
inserting a flexible insole into the shoe;
sensing force at first and second regions of the insole, and, responsive thereto, to generating respective first and second force measurements, the first and second regions corresponding to first and second regions of a sole of the shoe, respectively;
generating a first output responsively to the first force measurement surpassing a first minimum threshold value indicative of ground contact of the first region of the sole; and
generating a second output, different from the first output, responsively to the second force measurement surpassing a second minimum threshold value indicative of ground contact of the second region of the sole.

There is also provided, in accordance with an embodiment of the present invention, a method for use with a shoe worn by a subject, the method including:
inserting a flexible insole into the shoe;
generating first and second force measurements for first and second regions of the insole, respectively;
receiving a measured sequence of the first and second force measurements;
comparing the measured sequence with a predetermined sequence; and
generating a correspondence output responsively to a determination that the measured sequence corresponds to the predetermined sequence.

There is additionally provided, in accordance with an embodiment of the present invention, apparatus including:
a shoe;
a flexible insole integrated into the shoe, the insole shaped so as to define at least two independent chambers inflatable with a substance selected from the group consisting of: a gas and a liquid;
at least two pressure sensors, positioned remotely from the shoe;
a connector, including a proximal portion and a distal portion that are removably coupleable to one another, the proximal portion of which is physically coupled to a lateral surface of the shoe;
at least first and second proximal tubes, respective proximal ends of which are in fluid communication with respective ones of the chambers, and respective distal ends of which are in fluid communication with the proximal portion of the connector; and
at least first and second distal tubes, respective distal ends of which are in fluid communication with respective ones of the pressure sensors, and respective proximal ends of which are in fluid communication with the distal portion of the connector,
wherein the connector is configured such that when the proximal and distal portions thereof are coupled to one another, the first proximal tube is in fluid communication with the first distal tube, and the second proximal tube is in fluid communication with the second distal tube.

The present invention will be more fully understood from the following detailed description of preferred embodiments thereof, taken together with the drawings, in which:

### BRIEF DESCRIPTION OF THE DRAWINGS

FIG. 1 is a schematic illustration of a rehabilitation system, in accordance with an embodiment of the present invention;

FIG. 2 is an exemplary graph showing weight/force values for two types of shoes, in accordance with an embodiment of the present invention;

FIG. 3A is a schematic illustration of RF welding apparatus;

FIG. 3B is a schematic illustration of an insole made on the apparatus of FIG. 3A;

FIG. 4A is a schematic front view of a connector, and FIGS. 4B and 4C are schematic rear views of the connector when uncoupled and coupled, respectively, in accordance with an embodiment of the present invention; and

FIG. 5 is a schematic illustration of a rehabilitation shoe, in accordance with an embodiment of the present invention.

### DETAILED DESCRIPTION OF EMBODIMENTS

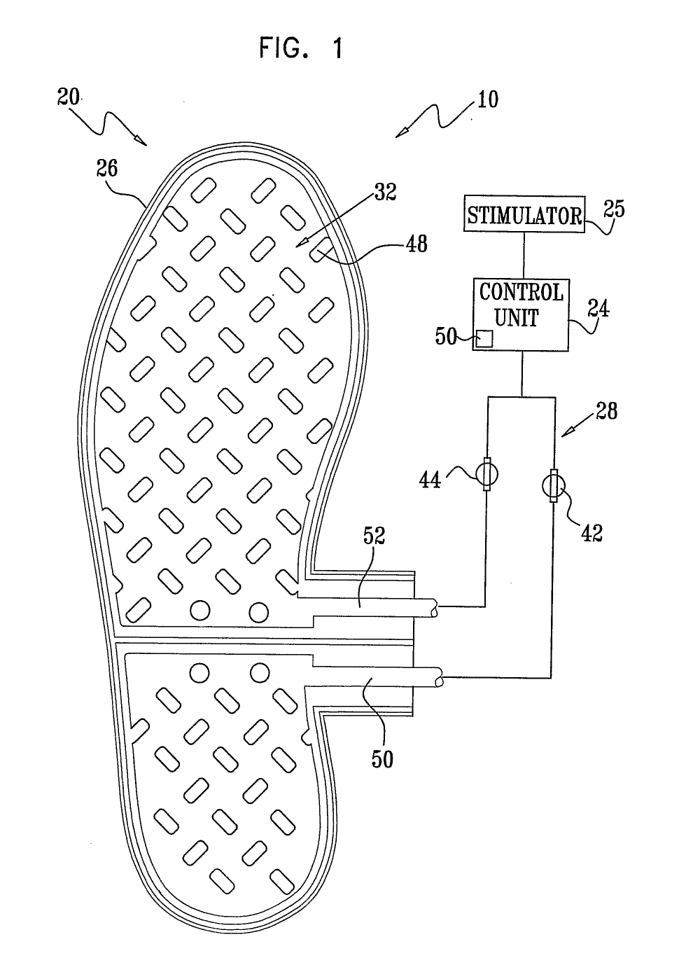

FIG. 1 is a schematic illustration of a rehabilitation system 10, in accordance with an embodiment of the present invention. Rehabilitation system 10 typically comprises an insole system 20, which is adapted to be inserted into a shoe worn by a subject, and a control unit 24. For some applications, control unit 24 is adapted to be worn by the user, such as by being strapped on a leg or arm of the user. Alternatively, control unit 24 comprises a general-purpose personal computer that is in wired or wireless communication with insole system 20.

Rehabilitation system 10 further comprises a stimulator 25 that provides feedback to the subject. The stimulator typically comprises an audio stimulator (e.g., a tone generator), a tactile stimulator (e.g., a vibrating unit), and/or a visual stimulator (e.g., a series of LED's, and/or a computer monitor). For some applications, stimulator 25 is integrated into control unit 24, while for other applications, the stimulator is a separate unit in wired or wireless communication with control unit 24.

Insole system 20 typically comprises a flexible insole 26 and at least one force sensor 28, adapted to generate a force measurement. For some applications, force sensor 28 is adapted to generate separate force measurements for a hindfoot region 30 and a forefoot region 32 of insole 26.

In an embodiment of the present invention, force sensor 28 comprises one or more pressure sensors, adapted to measure a pressure in insole 26 and generate the force measurement responsively to the measured pressure.

In an embodiment of the present invention, force sensor 28 comprises at least two pressure sensors 42 and 44, positioned remotely from insole 26. Insole 26 is shaped so as to define at least two independent chambers 46 and 48, which are inflatable with a gas or a liquid. Insole system 20 comprises at least two tubes 50 and 52, each of which is in fluid communication with a respective one of chambers 46 and 48, and with a respective one of pressure sensors 42 and 44. For some applications, insole 26 is shaped so as to define exactly two independent chambers that are operatively coupled to respective ones of the pressure sensors. For some applications, insole 26 is shaped so as to define a first one 46 of the chambers in hindfoot region 30 of the insole, and a second one 48 of the chambers in forefoot region 32 of the insole.

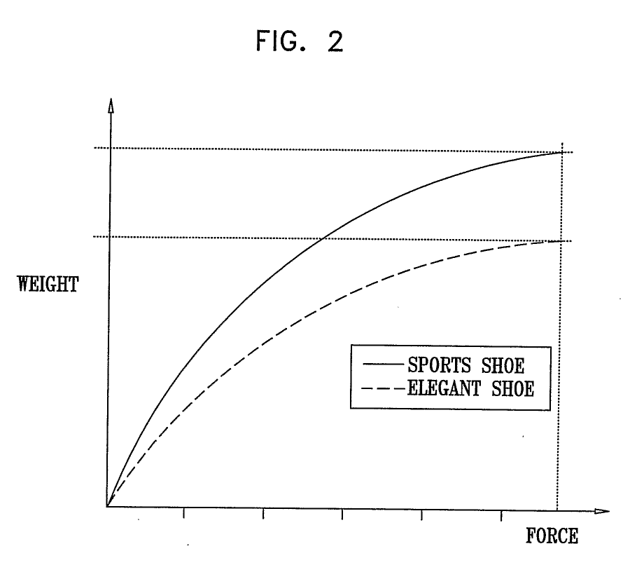

For some applications, rehabilitation system 10 utilizes techniques described in PCT Publication WO 2004/008095 to Avni et al., which is assigned to the assignee of the present application and is incorporated herein by reference. In particular, rehabilitation system 10 may utilize techniques described in the section thereof entitled “The Foot Force Sensor” (paragraphs [0032] through [0042]) and shown in FIGS. 1-2C thereof.

In an embodiment of the present invention, control unit 24 is adapted to receive the force measurement, and convert the force measurement to a weight measurement. Typically, the force measurement is initially measured as an analog signal generated in response to the deflection of a mechanical element. For example, the analog force measurement may be the typical 0.2-4.9 V output signal of a Motorola MPX4250D series piezoresistive transducer. Typically, an A/D converter converts the analog force measurement to a digital value, e.g., the A/D converter may output 1023 values. For some applications, control unit 24 comprises a memory 50, in which a look-up table is stored. The look-up table contains force-to-weight conversion values for a plurality of digital force measurement values. The control unit is adapted to use the look-up table to convert the force measurement to the weight measurement. For example, a digital value of 290-305 may represent a baseline normal inflation pressure for a European size 42 shoe, and the value 290 may thus be selected as the zero-point value, reflecting the application of no weight. In this example, a measured digital value 540 is converted to an applied weight value by subtracting the zero-point value (290), and converting the resulting difference of 250 into a weight of 59 kg in the hindfoot and 99 kg in the forefoot.

An application of this embodiment comprises presenting the post-conversion weight measurements to a therapist and/or to the subject, as many therapies are calibrated in terms of weight (e.g., to encourage the patient to put between 5 and 10 kilogram-force on the hindfoot).

For some applications, control unit 24 is adapted to convert the force measurement to the weight measurement at least in part responsively to an indication of whether the user is stepping or standing still. For some applications, control unit 24 comprises memory 50, in which a look-up table is stored. The look-up table contains, for a plurality of force measurement values, stepping force-to-weight conversion values and standing-still force-to-weight conversion values. Control unit 24 is adapted to use the look-up table to convert the force measurement to the weight measurement.

For some applications in which force sensor 28 is adapted to generate separate force measurements for hindfoot and forefoot regions 30 and 32 of insole 26, control unit 24 is adapted to convert the hindfoot force measurement to a hindfoot weight measurement, and the forefoot force measurement to a forefoot weight measurement.

The inventors have observed that the force measurements generated by insole 26 upon any given application of weight vary responsively to one or more shoe/foot characteristics, such as the type and size of shoe the subject is wearing, whether the subject is an amputee fitted with a lower-leg prosthesis or a prosthetic foot, whether a shoe insert has been inserted in the shoe (such as an orthotic insert, e.g., an ankle foot orthosis (AFO)), or other characteristics of the foot, shoe, or foot/shoe interface that affect the force measurements. (In the present application, including in the claims, the size of the shoe is to be understood as being equivalent to the size of the insole inserted in the shoe.) Without the use of techniques described herein, this variability generally necessitates the performance of a calibration procedure for each subject while wearing his or her shoes, in order to achieve acceptable measurement accuracy.

In an embodiment of the present invention, control unit 24 is adapted to receive the force measurement, and utilize the force measurement in a calculation that is performed at least in part responsively to at least one shoe/foot characteristic that is entered into control unit 24 by the subject or a therapist. Typically, the characteristic of the shoe includes a size of the shoe, a type of the shoe, both a size and a type of the shoe, whether the subject is an amputee fitted with a lower-leg prosthesis or a prosthetic foot, whether a shoe insert has been inserted in the shoe, or another characteristic of the foot, shoe, or foot/shoe interface that affects the force measurement. For some applications, control unit 24 is adapted to convert the force measurement to a weight measurement at least in part responsively to the shoe/foot characteristic. The use of the techniques of this embodiment generally enables accurate utilization of force measurements generated using insole 26 in the subject's own shoes, without the need for performing a calibration procedure for each subject while wearing his or her shoes.

Reference is made to FIG. 2, which is an exemplary graph showing weight/force values for two types of shoes, in accordance with an embodiment of the present invention. The exemplary graph shows that any given force measurement results in a different weight measurement for the two types of shoes. Knowledge of which type of shoe the subject is wearing enables accurate conversion of measured force values to clinically-relevant weight values.

In an embodiment of the present invention, control unit 24 comprises memory 50, in which a look-up table is stored. The look-up table is populated with force-to-weight conversion values for a plurality of force measurement values and a plurality of values of the shoe/foot characteristic. Control unit 24 is adapted to use the look-up table to convert the force measurement to the weight measurement.

For some applications, the look-up table is populated using the following calibration procedure, typically during manufacture of rehabilitation system 10. Typically, the calibration procedure is performed separately for several common types (e.g., sport shoes, elegant shoes, casual shoes) and sizes of shoes. Insole 26 is inserted into the test shoe, and a plurality of weights (e.g., between 1 and 125 kg) are applied to the inside of the shoe, each typically for several seconds. The amount of weight applied is typically measured using a conventional force platform upon which the shoe has been placed. Typically, separate measurements are made for the forefoot and hindfoot regions of the insole. Alternatively or in combination with shoe type/size, the calibration procedure is performed separately for subjects with and without a prosthesis, and/or with and without a shoe insert inserted in the shoe.

In an embodiment of the present invention, force sensor 28 is adapted to generate separate force measurements for hindfoot and forefoot regions 48 and 52 of the insole, and control unit 24 is adapted to utilize the hindfoot force measurement and the forefoot force measurement in the calculation that is performed at least in part responsively to the shoe/foot characteristic. For some applications, control unit 24 is adapted to convert, at least in part responsively to the shoe/foot characteristic, the hindfoot force measurement to a hindfoot weight measurement, and the forefoot force measurement to forefoot weight measurement.

For some applications, control unit 24 comprises memory 50, in which a look-up table is stored. The look-up table contains, for a plurality of force measurement values and a plurality of values of the shoe/foot characteristic, hindfoot force-to-weight conversion values and forefoot force-to-weight conversion values. Control unit 24 is adapted to use the look-up table to convert the hindfoot and forefoot force measurements to the hindfoot and forefoot weight measurements, respectively.

For some applications, control unit 24 is adapted to perform the calculation at least in part responsively to an indication of whether the user is stepping or standing still. For some applications, control unit 24 is adapted to convert the force measurement to a weight measurement, at least in part responsively to the indication of whether the user is stepping or standing still. For some applications, control unit 24 comprises memory 50, in which a look-up table is stored. The look-up table contains, for a plurality of force measurement values and a plurality of values of the shoe/foot characteristic, stepping force-to-weight conversion values and standing-still force-to-weight conversion values. Control unit 24 is adapted to use the look-up table to convert the force measurement to the weight measurement.

In an embodiment of the present invention, control unit 24 is adapted to be portable, and to be physically coupled to and carried on a limb of the subject. For some applications, control unit 24 comprises memory 50, in which a look-up table is stored. The look-up table contains force-to-weight conversion values for a plurality of values of at least one shoe/foot characteristic. Control unit 24 is adapted to receive an indication of a value of the shoe/foot characteristic, receive the force measurement, and use the look-up table to convert the force measurement to a weight measurement, at least in part responsively to the value of the shoe/foot characteristic.

In an embodiment of the present invention, a calibration procedure comprises making one or more first-disposition force measurements while the subject is in a first disposition, and making one or more second-disposition force measurements while the subject is in a second disposition. The calibration procedure may be used, for example, to fill a look-up table for converting force measurements to weight measurements, as described herein. For some applications, force measurements in the calibration procedure are made at a larger number of dispositions, typically as the subject moves from an original disposition to a final disposition. In one embodiment, the subject moves smoothly from the original disposition to the final disposition, while control unit 24 makes intermittent force measurements (e.g., every 1-10, 10-100, or 100-1000 ms). In another embodiment, the subject is instructed to assume each of the discrete dispositions between the original and the final dispositions, and the force measurement is made when the subject is in each of the intermediate dispositions. In this case, the number of intermediate dispositions is usually limited to under 10, e.g., 3-5, for the ease of the subject.

For various applications, the original and final dispositions may be, respectively (or in reverse order):
weight on hindfoot, weight on forefoot (measured statically, or while walking or running);
weight on left foot, weight on right foot;
weight balanced between both feet, weight mostly or entirely on one foot; and/or
sitting, standing.

As appropriate for a given subject, measurements may be made while the subject is using or not using a shoe insert, such as an orthosis (e.g., an ankle-foot orthosis) or a prosthetic device (e.g., a lower-leg prosthesis or a prosthetic foot). Similarly, in order to calibrate the insole for use outside of the laboratory, whereby to help the subject prepare for normal activities of daily living, the calibration procedure may be performed on different simulated (or real) surfaces, e.g., pavement, wood floor, or carpet.

In an embodiment of the present invention, a method is provided for calibrating insole 26 for a particular subject. The insole is inserted into the shoe (typically the subject's own shoe), and thereafter the subject inserts his or her foot into the shoe. The shoe is weighed one or more times while the subject is applying weight to the shoe, so as to generate a set of one or more measured weight values. (Thus, the weighing of the shoe generates a measurement indicating the total force applied by the shoe to a weighing device, due both to the mass of the shoe and to any force applied by the subject to the insole.) While the shoe is weighed, a force applied by the foot on insole 26 is measured, so as to generate a set of one or more measured force values. Responsively to the measured weight values and the measured force values, a look-up table is generated containing force-to-weight conversion values applicable to the shoe.

For some applications, the shoe is weighed and the force applied by the foot is measured a first number of one or more times, and the look-up table is generated containing a second number of the force-to-weight conversion values, the second number greater than the first number. For some applications, the force applied by the foot is measured exactly once.

For some applications, the measured weights and forces are fit to a pre-calibrated force-to-weight curve, typically by maintaining relative slopes along the pre-calibrated curve. Such fitting enables satisfactorily complete population of the look-up table based on only a single or a small number of calibration measurements of the subject. Typically, a single pre-calibrated force-to-weight curve is used for shoes having different characteristics, i.e., the shape of the curve is not dependent on shoe characteristics. Alternatively, for some applications, a plurality of pre-calibrated force-to-weight curves are provided for shoes having different characteristics (e.g., type and/or size), and the curve appropriate for the subject's shoe is selected.

For some applications, the calibration method further utilizes a pre-populated calibration-shoe table indicating force-to-weight conversion values for a calibration shoe. The calibration-shoe table includes at least two portions, including a calibration-shoe high-force portion and a calibration-shoe low-force portion. The calibration-shoe high-force portion includes higher force values than the calibration-shoe low-force portion. At least one numerical relationship is identified that relates values in the calibration-shoe high-force portion to values in the calibration-shoe low-force portion. The look-up table includes at least two portions, including a look-up table high-force portion and a look-up table low-force portion, the look-up table high-force portion including higher force values than the look-up table low-force portion. The look-up table is generated by setting values in the look-up table responsively to the identified relationship between the values in the calibration-shoe high-force portion and the values in the calibration-shoe low-force portion.

For some applications, the numerical relationship includes a relationship between (a) a characteristic slope of the values in the calibration-shoe high-force portion and (b) a characteristic slope of the values in the calibration-shoe low-force portion. The look-up table is generated responsively to the relationship between the characteristic slopes.

In an embodiment of the present invention, a method is provided for calibrating insole 26. In this embodiment, chambers 46 and 48 are typically inflatable with a gas. Insole 26 is inserted into the shoe, and thereafter the subject inserts his or her foot into the shoe. Respective pressures are measured in each of the chambers after the foot has been inserted, while weight of the subject is not applied to the shoe. Each of the chambers is inflated until respective desired pressures are obtained in the chambers.

In an embodiment of the present invention, a method is provided for calibrating insole 26 for a particular subject wearing the subject's own shoe and, optionally, a prosthesis and/or orthotic insert. The method utilizes a pre-populated look-up table containing force-to-weight conversion values for a typical foot and shoe. The look-up table is typically pre-populated by the manufacturer by taking averages of calibration measurements of a plurality of typical subjects wearing typical shoes. Insole 26 is inserted into the subject's shoe, and thereafter the subject inserts his or her natural or prosthetic foot into the shoe. If the subject uses an orthotic insert, the insert is inserted into the shoe after inserting insole 26.

The shoe is weighed one or more times while the subject is applying weight to the shoe, so as to generate a set of one or more measured weight values. For example, the shoe is weighed five or fewer times, such as three or fewer times, e.g., exactly two times. Typically, the subject is instructed to apply, during each of the one or more times, a weight to the shoe equal to a respective weight value of one of the force-to-weight conversion values in the pre-populated look-up table. While the shoe is weighed, a force applied by the foot on insole 26 is measured, so as to generate a set of one or more measured force values. The force-to-weight conversion values of the pre-populated look-up table are corrected responsively to the measured weight values and the measured force values. The force-to-weight conversion values corresponding to measured weights are corrected by substituting the measured force-to-weight value for the pre-populated force-to-weight value, and the remaining force-to-weight values are corrected based on the pre-populated force-to-weight values and a mathematical relationship (e.g., one or more ratios) between the measured force-to-weight values and the pre-populated force-to-weight values.

For some applications, the weight values are measured using a conventional scale, such as a consumer scale marketed for home use. Alternatively, a scale with an output signal is coupled to insole system 20. In either case, the subject applies the instructed weight to the shoe by placing the subject's shoe, while worn, on the scale and adjusting the weight applied to the shoe until the weight value displayed by the scale and/or system 20 equals the desired value. The subject applies the remainder of his or her weight to the subject's other foot, a handrail, a chair, or another convenient surface.

In an embodiment of the present invention, force sensor 28 comprises at least two force sensors adapted to sense force at respective locations of insole 26, and, responsive thereto, to generate respective force measurements. Rehabilitation system 10 comprises a stimulator 60, adapted to provide feedback to the subject. Control unit 24 is adapted to receive the force measurements; perform an analysis of a gait of the subject at least in part responsively to the force measurements; and responsively to the gait analysis, drive stimulator 60 to configure the feedback to guide the subject to modify one or more respective forces applied by the subject to a subset of the respective locations of insole 26, the subset containing fewer than all of the locations of insole 26.

In an embodiment of the present invention, control unit 24 is adapted to detect a temporal pattern of footsteps of the subject. In this embodiment, control unit 24 typically uses the force measurements only for sensing ground contact of the shoe. The control unit typically does not analyze the magnitude of the force measurements beyond determining that a minimum threshold has been surpassed indicative of ground contact. In other words, the minimum threshold is not itself being used as a diagnostic tool, e.g., for determining whether the subject is applying enough weight to the foot.

In this embodiment, force sensor 28 is adapted to generate first and second force measurements for first and second regions of insole 26, respectively. For example, the first and second regions may include hindfoot region 30 and forefoot region 32. Control unit 24 is adapted to drive stimulator 25 to generate a first output responsively to the first force measurement surpassing a first minimum threshold value indicative of ground contact of a first region of the sole, and generate a second output, different from the first output, responsively to the second force measurement surpassing a second minimum threshold value indicative of ground contact of a second region of the sole. For some applications, the first and second minimum threshold values are each indicative of a threshold weight equal to between 3% and 15% of a weight of the subject, such as between 5% and 10% of the subject's weight.

For some applications, the first and second outputs include audible outputs. For some applications, the outputs are tones having different pitches, or tones having a different number of pulses. For example, control unit 24 may generate a single tone upon detection of hindfoot ground contact, and a double tone upon detection of forefoot ground contact. As a result, if the subject walks with proper form, the control unit generates a single tone, followed by a short pause, followed by a double tone, indicating that the hindfoot is properly malting ground contact prior to the forefoot during a step. Alternatively or additionally, the outputs are tactile signals (e.g., vibrations), and/or visual signals (e.g., stimulator 25 comprises one or more LED's, and/or rehabilitation system 10 comprises a computer monitor).

In an embodiment of the present invention, control unit 24 is adapted to determine whether a sequence of measurements from force sensor 28 matches a predetermined sequence (or does not match the sequence). Responsively to the determination, control unit 24 generates an output, such as an audible output. As a result, when the subject walks with proper heel-toe form, he or she detects the output.

In this embodiment, force sensor 28 is adapted to generate first and second force measurements for first and second regions of insole 26, respectively. For example, the first and second regions may include hindfoot region 30 and forefoot region 32. Alternatively, for some applications, force sensor 28 is adapted to generate first, second, and third force measurements for three respective regions of insole 26. For example, the three regions may include heel, lateral, and medial regions. Alternatively, for some applications, force sensor 28 is adapted to generate first, second, third, and fourth force measurements for four respective regions of insole 26, such as heel, lateral, medial, and great toe regions.

In an embodiment of the present invention, stimulator 25 comprises a tactile stimulator adapted to apply a vibration signal to a site on the leg of the subject, typically other than the foot. The site typically includes an area of skin (e.g., a dermatome from L1 through L5) supplied by a nerve, such as a lateral femoral cutaneous nerve, a medial femoral cutaneous nerve, a cutaneous branch of an obturator nerve, a branch of a posterior femoral cutaneous nerve, a lateral sural cutaneous nerve, a saphenous nerve, or a cutaneous branch of a common fibular nerve.

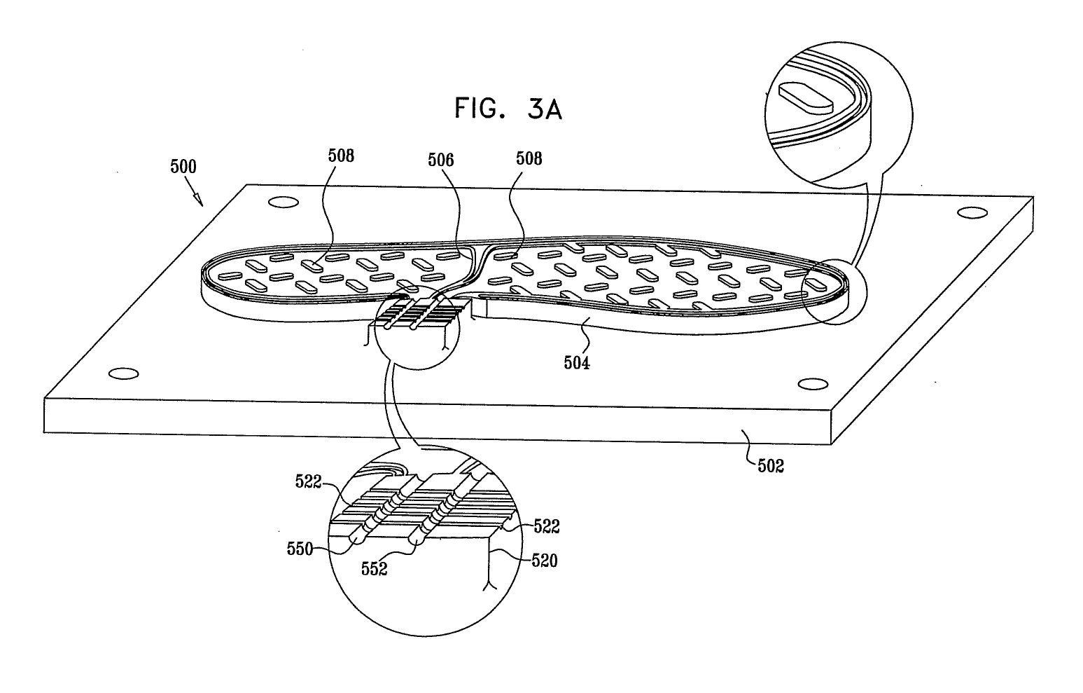

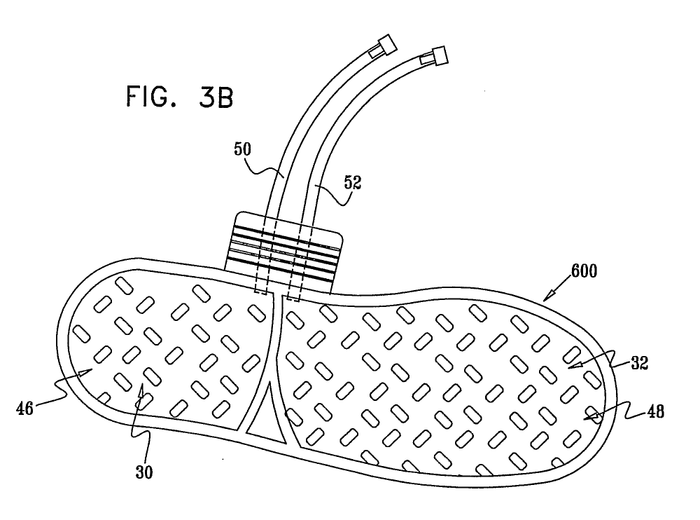

Reference is now made to FIGS. 3A and 3B, which are schematic illustrations of RF welding apparatus 500 and an insole 600 made on apparatus 500, in accordance with an embodiment of the present invention. Apparatus 600 comprises a metal base 502, having raised metallic elements 504 that define the outside edge of insole 600, metallic spacers 508 that define contact regions between upper and lower surfaces of insole 600, and a metallic chamber divider 506 that defines the division between chambers 46 and 48 of insole 600.

For clarity, two steps are defined below for RF welding. These steps may be performed separately (e.g., step 1 before step 2, or vice versa), or they may be performed during a single welding operation. Similarly, it is noted that the scope of the present invention is not limited to RF welding, and includes other suitable techniques that are known in the art (e.g., ultrasonic welding, and/or adhesives). Similarly, although embodiments are described hereinbelow with respect to the use of polyurethane, other suitable materials (e.g., PVC) may also be substituted, mutatis mutandis.

Step 1: In an embodiment, a polyurethane sheet is placed over a tube-mounting block 520 projecting out of metal base 502. The sheet covers longitudinal channels 522 and tube-supporting channels 550 and 552 of block 520. Tubes 50 and 52 are aligned, over the polyurethane sheet, in tube-supporting channels 550 and 552, respectively. Metal bars are typically inserted (or pre-inserted) into tubes 50 and 52, in order to support the tubes mechanically from within, and/or to promote a desired heating profile of the tubes. While for some applications the metal bars are connected to an RF power supply, the scope of the present invention includes not using the metal bars as RF electrodes. (For some applications, non-metallic bars are used.) A second polyurethane sheet is placed over the tubes, and RF welding binds the first and second polyurethane sheets to each other, around the tubes.

The inventors hypothesize that during the RF welding, longitudinal channels 522 provide extra space for enhancing the bonding between the tubes and the surrounding polyurethane sheets. For example, temporarily melted portions of the sheets and the tubes have extra space to mix, and then bond when cooled, due to the presence of the longitudinal channels. In this manner, a better pressure seal is attained in insole 600 at the locations of insertions of tubes 50 and 52.

The scope of the present invention includes omitting the longitudinal channels.

Step 2: In an embodiment, the two polyurethane sheets are placed above elements 504, spacers 508, and chamber divider 506. RF welding binds the sheets to each other, so as to provide a two-chamber insole.

In RF welding embodiments, elements 504, spacers 508, chamber divider 506, and block 520 all typically serve as electrodes.

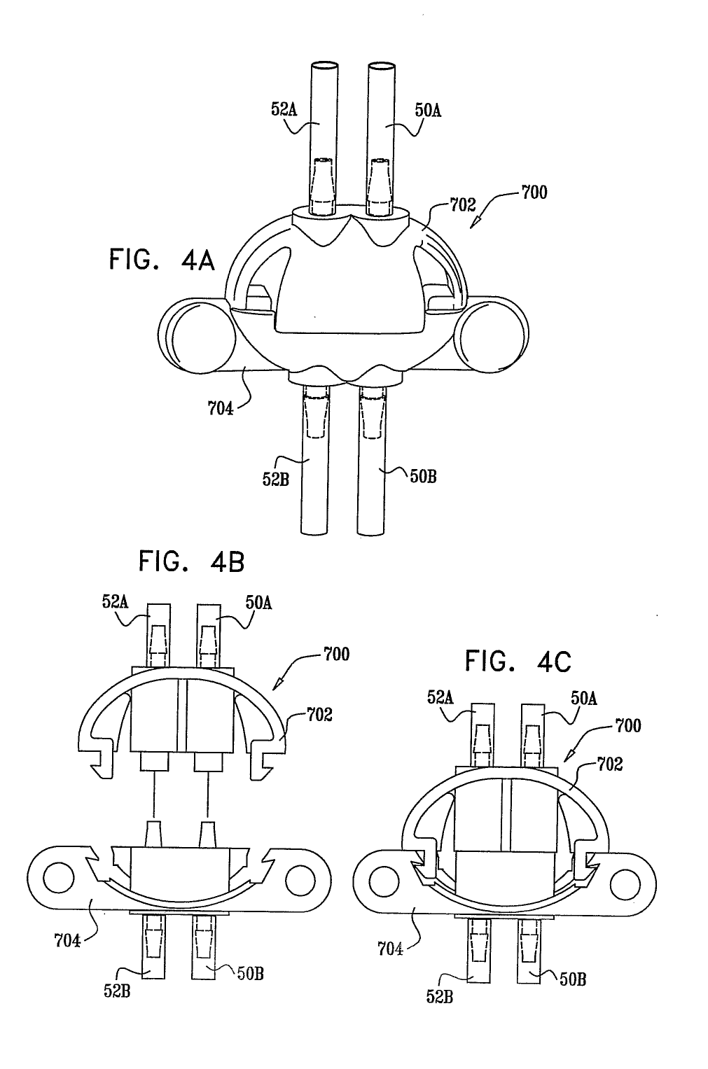

Reference is made to FIG. 4A, which is a schematic front view of a connector 700, and FIGS. 4B and 4C, which are schematic rear views of the connector when uncoupled and coupled, respectively, in accordance with an embodiment of the present invention. Connector 700 is used to simultaneously removably couple a distal (with respect to the insole) portion 50A of tube 50 to a proximal portion 50B thereof, and a distal portion 52A of tube 52 to a proximal portion 52B thereof. Connector 700 comprises a distal element 702, which is coupled to distal portions 50A and 52A of tubes 50 and 52, respectively, and a proximal element 704, which is coupled to proximal portions 50B and 52B of tubes 50 and 52, respectively. Elements 702 and 704 are configured to removably engage one another. In the embodiment shown in the figures (most clearly in FIG. 4B), element 702 is disengaged from element 704 by squeezing the sides of element 702. Other mechanisms for coupling the elements together will be evident to those skilled in the art who have read the present application, and are within the scope of the present invention.

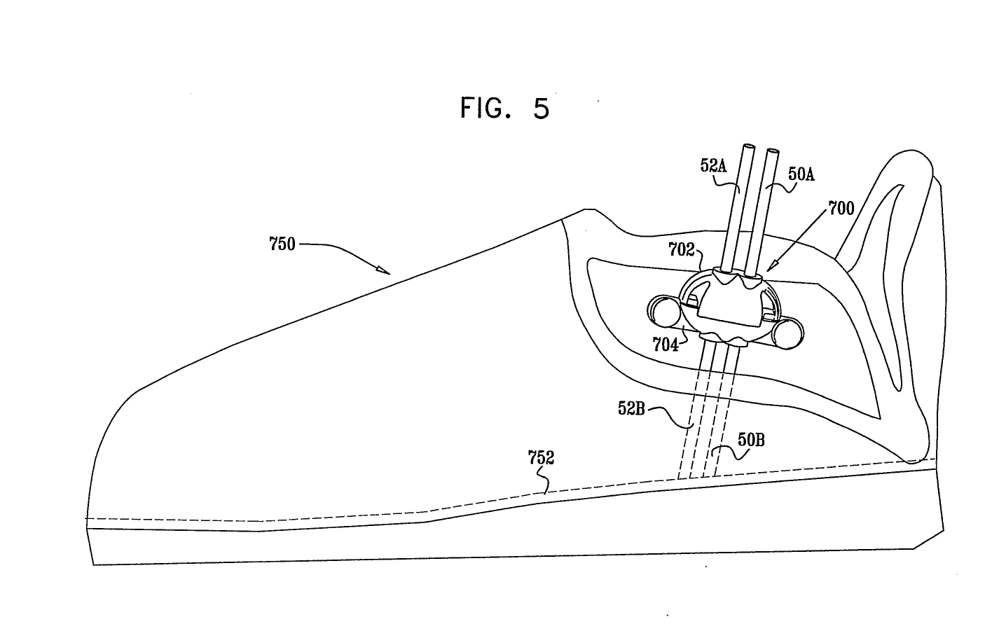

Reference is made to FIG. 5, which is a schematic illustration of a rehabilitation shoe 750, in accordance with an embodiment of the present invention. Shoe 750 comprises a flexible insole 752, which is integrated with the shoe. Insole 752 is generally similar to insole 26, described hereinabove with reference to FIGS. 1 and 3B. For some applications, shoe 750 comprises connector 700, described hereinabove with reference to FIGS. 4A and 4B. At least a portion of proximal portions 50B and 52B of tubes 50 and 52 are typically contained within a side wall of the shoe.

References to a “foot” in the claims are to be understood to include both a natural foot and a prosthetic foot, unless otherwise indicated or clearly implied by context.

The scope of the present invention includes embodiments described in the following patent references, which are assigned to the assignee of the present application and are incorporated herein by reference. In an embodiment, techniques and apparatus described in one or more of the following patent references are combined with techniques and apparatus described herein:
U.S. Provisional Patent Application 60/526,814, filed Dec. 3, 2003;
PCT Publication WO 01/36051 to Avni, filed Oct. 27, 2000; and/or
U.S. Pat. No. 6,273,863 to Avni et al.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1-37. (canceled)

38. Apparatus for use with a shoe worn by a subject, the apparatus comprising: an insole system, adapted to be inserted into the shoe, the insole system comprising a flexible insole, and at least two force sensors adapted to sense force at respective locations of the insole, and, responsive thereto, to generate respective force measurements; a stimulator, adapted to provide feedback to the subject; and a control unit, adapted to: receive the force measurements, perform an analysis of a gait of the subject at least in part responsively to the force measurements, and responsively to the gait analysis, drive the stimulator to configure the feedback to guide the subject to modify one or more respective forces applied by the subject to a subset of the respective locations of the insole, the subset containing fewer than all of the locations of the insole.

39. Apparatus for use with a shoe worn by a subject, the apparatus comprising: an insole system, adapted to be inserted into the shoe, the insole system comprising a flexible insole and at least one force sensor, adapted to generate a force measurement; and
a portable control unit, adapted to be physically coupled to and carried on a limb of the subject, the control unit comprising a memory, in which a look-up table is stored, the look-up table containing force-to-weight conversion values for a plurality of values of at least one shoe/foot characteristic, and the control unit adapted to:
receive an indication of a value of the shoe/foot characteristic,
receive the force measurement,
and use the look-up table to convert the force measurement to a weight measurement, at least in part responsively to the value of the shoe/foot characteristic.

40. The apparatus according to claim 39, wherein the force measurement is expressed as a digital value, and wherein the force sensor is adapted to convert a measured analog signal into the digital force measurement.

41. (canceled)

42. (canceled)

43. The apparatus according to claim 39, wherein the shoe/foot characteristic includes a type of the shoe and a size of the shoe, and wherein the look-up table contains the force-to-weight conversion values for a plurality of shoe types and shoe sizes.

44. (canceled)

45. (canceled)

46. A method for calibrating a flexible insole, the method comprising:
inserting the insole into a shoe;
inserting a foot of a subject into the shoe after the insole has been inserted;
weighing, one or more times, the shoe while the subject is applying weight to the shoe, so as to generate a set of one or more measured weight values;
while weighing the shoe, measuring a force applied by the foot on the insole, so as to generate a set of one or more measured force values; and
responsively to the measured weight values and the measured force values, generating a look-up table containing force-to-weight conversion values applicable to the shoe.

47. The method according to claim 46,
wherein weighing the shoe and measuring the force applied by the foot comprise weighing the shoe and measuring the force applied by the foot a first number of one or more times, and
wherein generating the look-up table comprises generating the look-up table containing a second number of the force-to-weight conversion values, the second number greater than the first number.

48. (canceled)

49. The method according to claim 47, wherein generating the look-up table comprises fitting the measured weight and force values to a predetermined curve, in order to generate the second number of the force-to- weight conversion values.

50. The method according to claim 47, comprising:
generating for a calibration shoe a calibration-shoe table indicating force-to- weight conversion values, the calibration-shoe table including at least two portions, the at least two portions including a calibration-shoe high-force portion and a calibration-shoe low-force portion, the calibration-shoe high-force portion including higher force values than the calibration-shoe low-force portion; and
identifying a numerical relationship relating values in the calibration-shoe high-force portion to values in the calibration-shoe low-force portion,
wherein the look-up table includes at least two portions, the at least two portions including a look-up table high-force portion and a look-up table low-force portion, the look-up table high-force portion including higher force values than the look-up table low-force portion, and
wherein generating the look-up table comprises setting values in the look-up table responsively to the identified relationship between the values in the calibration- shoe high-force portion and the values in the calibration-shoe low-force portion.

51. The method according to claim 50,
wherein the numerical relationship includes a relationship between (a) a characteristic slope of the values in the calibration-shoe high-force portion and (b) a characteristic slope of the values in the calibration-shoe low-force portion, and
wherein generating the look-up table comprises generating the look-up table responsively to the relationship between the characteristic slopes.

52. The method according to claim. 46, wherein generating the look-up table comprises:
providing a look-up table pre-populated with a plurality of force-to-weight conversion values applicable to a typical foot and a typical shoe; and
correcting the conversion values of the pre-populated look-up table responsively to the measured weight values and the measured force values.

53. The method according to claim 52, wherein weighing comprises instructing the subject, during each of the one or more times, to apply a respective weight to the shoe equal to a respective weight value of one of the force-to-weight conversion values in the pre-populated look-up table.

54. (canceled)

55. A method for calibrating a flexible insole shaped so as to define at least two independent chambers inflatable with a gas, comprising:
inserting the insole into a shoe;
inserting a foot of a subject into the shoe after the insole has been inserted;
measuring respective pressures in each of the chambers after the foot has been inserted, while weight of the subject is not applied to the shoe; and
inflating each of the chambers until respective desired pressures are obtained in the chambers.

103. A method for use with a shoe worn by a subject, the method comprising:
inserting a flexible insole into the shoe;
generating a force measurement of a force applied to the insole;
physically coupling a portable control unit to a limb of the subject, such that the control unit is carried on the limb;
receiving, by the control unit, an indication of a value of at least one shoe/foot characteristic; and
converting, by the control unit, the force measurement to a weight measurement, at least in part responsively to the value of the shoe/foot characteristic, using a look-up table containing force-to-weight conversion values for a plurality of values of the at least one shoe characteristic.

104. The method according to claim 103, wherein the force measurement is expressed as a digital value, and wherein generating the force measurement comprises measuring an analog signal, and converting the analog signal into the digital force measurement.

105. The method according to claim 103, wherein the shoe/foot characteristic includes a type of the shoe, and wherein converting comprises using the look-up table containing the force-to-weight conversion values for a plurality of shoe types.

106. The method according to claim 103, wherein the shoe/foot characteristic includes a size of the shoe, and wherein converting comprises using the look-up table containing the force-to-weight conversion values for a plurality of shoe sizes.

107. The method according to claim 103, wherein the shoe/foot characteristic includes a type of the shoe and a size of the shoe, and wherein converting comprises using the look-up table containing the force-to-weight conversion values for a plurality of shoe types and shoe sizes.

108. (canceled)

109. (canceled)

110. A method for use with a shoe worn by a subject, the method comprising:
inserting a flexible insole into the shoe;
sensing force at first and second regions of the insole, and, responsive thereto, to generating respective first and second force measurements, the first and second regions corresponding to first and second regions of a sole of the shoe, respectively;
generating a first output responsively to the first force measurement surpassing a first minimum threshold value indicative of ground contact of the first region of the sole; and
generating a second output, different from the first output, responsively to the second force measurement surpassing a second minimum threshold value indicative of ground contact of the second region of the sole.

111. The method according to claim 110, wherein the first and second minimum threshold values are each indicative of a threshold weight equal to between 5% and 10% of a weight of the subject.

112. The method according to claim 110, wherein the first and second regions of the insole include hindfoot and forefoot regions of the insole, respectively, and wherein generating the first and second force measurements comprises generating the first and second force measurements for the hindfoot and forefoot regions of the insole, respectively.

113. The method according to claim 110, wherein the first and second outputs include first and second audible outputs, respectively, and wherein generating the first and second outputs comprises generating the first and second audible outputs.

114. A method for use with a shoe worn by a subject, the method comprising: inserting a flexible insole into the shoe;
generating first and second force measurements for first and second regions of the insole, respectively;
receiving a measured sequence of the first and second force measurements;
comparing the measured sequence with a predetermined sequence; and
generating a correspondence output responsively to a determination that the measured sequence corresponds to the predetermined sequence.

115. The method according to claim 114, comprising generating a non-correspondence output responsively to a determination that the measured sequence does not correspond to the predetermined sequence.

116. The method according to claim 114, wherein generating the correspondence output comprises generating an audible correspondence output.

117. The method according to claim 114 any one of claims 114-116, wherein first and second regions of the insole include hindfoot and forefoot regions of the insole, respectively, and generating the first and second force measurements comprises generating the first and second force measurements for the hindfoot and forefoot regions of the insole, respectively.

118. The method according to claim 117, wherein the predetermined sequence is indicative of heel-toe walking, and wherein generating the correspondence output comprises generating the correspondence output responsively to the determination that the measured sequence is indicative of the heel-toe walking.

119. (canceled)
